(19) United States
(12) Patent Application Publication
(10) Pub. No.: US 2024/0383823 A1
(43) Pub. Date: Nov. 21, 2024

(54) PROCESS FOR MANUFACTURING A MONOCYCLIC AROMATIC COMPOUND

(71) Applicant: Koch Technology Solutions, LLC, Wichita, KS (US)

(72) Inventors: Brant Lane Aggus, Corpus Christi, TX (US); Daniel Travis Shay, Chadds Ford, PA (US)

(21) Appl. No.: 18/693,527

(22) PCT Filed: Oct. 21, 2022
(86) PCT No.: PCT/IB2022/060127

§ 371 Date: Mar. 20, 2024

Related U.S. Application Data
(60) Provisional application No. 63/279,073, filed on Nov. 13, 2021.

(51) Int. Cl. C07C 1/20; C07C 1/12

(52) U.S. Cl. CPC C07C 1/20; C07C 1/12; C07C 2529/40

(57) ABSTRACT

A process for manufacturing a monocyclic aromatic compound is disclosed. The process comprises contacting an aluminosilicate catalyst with an oxygen-containing organic molecule in a reactor to produce the monocyclic aromatic compound and in situ-generated hydrogen gas, and introducing CO₂ into the reactor and allowing the CO₂ to react with the in situ-generated hydrogen gas to form additional monocyclic aromatic compound.

### RELATED APPLICATIONS

This application claims priority to U.S. Provisional Patent Application No. 63/279,073, filed Nov. 13, 2021, the entirety of which is incorporated herein by reference.

### FIELD OF THE INVENTION

The present disclosure relates to a process for the high-selectivity conversion of oxygen-containing organic molecules to monocyclic aromatic compounds, such as benzene, toluene or xylene, or mixtures thereof.

### BACKGROUND

Monocyclic aromatic compounds, such as BTX (mixtures of benzene, toluene, and xylene isomers), are important chemicals in the petroleum refining, petrochemical, and specialty chemical industries. A number of industrial processes for synthetically producing BTX compounds are known. For example, BTX and related C9/C10 aromatic compounds can be formed from oxygen-containing organic molecules, such as alcohols. U.S. Pat. No. 8,962,902B2 and U.S. Pat. No. 9,388,092B2 describe processes for converting alcohols to BTX compounds in the presence of zeolite catalysts at elevated temperatures. However, these high temperatures can result in detrimental effects to the zeolite catalyst. For example, coke may be formed and deposited on the catalyst surface and pores, effectively blocking active sites on the catalyst and reducing activity.

Accordingly, there is a need for a process for the high-selectivity conversion of oxygen-containing organic molecules to monocyclic aromatic compounds, thereby increasing the yield without premature catalyst deactivation to the extent found in the prior art.

### SUMMARY OF THE INVENTION

In accordance with a first aspect of the invention, there is provided a process for manufacturing at least one monocyclic aromatic compound, the process comprising contacting an aluminosilicate catalyst with an oxygen-containing organic molecule in a reactor to produce the monocyclic aromatic compound and in situ-generated hydrogen gas; and introducing CO2 into the reactor and allowing the CO2 to react with the in situ-generated hydrogen gas to form additional monocyclic aromatic compound.

An advantage of the process of the first aspect is that it produces a higher yield of product per unit of oxygen-containing organic molecule. This is because not only is the oxygen-containing organic molecule converted into product, but the CO2 introduced into the reactor reacts with hydrogen released from the conversion of the oxygen-containing molecule (the in situ-generated hydrogen gas), to produce additional monocyclic aromatic compound in an efficient one-pot synthesis.

At least a portion of the CO2 and in situ-generated hydrogen gas may react to form carbon monoxide. The process may further comprise allowing the carbon monoxide to contact the catalyst.

In accordance with a second aspect of the invention, there is provided a process for manufacturing at least one monocyclic aromatic compound, the process comprising contacting an aluminosilicate catalyst with an oxygen-containing organic molecule in a reactor to produce the monocyclic aromatic compound and in situ-generated hydrogen gas, introducing CO2 into the reactor and allowing the CO2 to react with the in situ-generated hydrogen gas to form carbon monoxide, and allowing the carbon monoxide to contact the catalyst.

An advantage of the processes of the first and second aspects is that they reduce coking of the catalyst, which prolongs the catalyst lifetime. This is achieved by the carbon monoxide coordinating with the most acidic sites of the catalyst to inhibit formation of the coke precursors.

The process may further comprise adding a further weakly-coordinating compound to the reactor, wherein the weakly-coordinating compound is a compound that reduces the H—O bond frequency of the aluminosilicate catalyst by about 1 to 300 cm-1 as measured by FT-IR.

The further weakly-coordinating compound may be selected from the group consisting of carbon monoxide, acetonitrile, dimethylsulfoxide, tetrahydrofuran, acetone, pyridine and tetrahydrothiophene. These are all commercially available organic compounds of relatively low cost which weakly coordinate to aluminosilicate catalysts and which may be included in the reactor to enhance the longevity of the catalyst. Preferably the further weakly-coordinating compound is carbon monoxide.

The CO2 that is introduced into the reactor may be derived from a biological, combustion, or chemical transformation process. An advantage of this feature of the process is that CO2 waste streams from biological sources (e.g. fermentation processes, such as processes for producing bioethanol from corn), combustion processes (e.g. coal and gas fired power stations), or industrial processes (e.g. fertiliser or cement production) can be used as a feedstock in the process and converted into a value-added product.

The oxygen-containing organic molecule may be derived from a fermentation process. The fermentation process may be a process for producing a C1-C4 alcohol from a grain crop, such as corn. An advantage of this feature is that it enables waste streams obtained from biological fermentation processes to be used as an inexpensive feedstock, reducing waste and generating a valuable product. An example of a fermentation process is a fermentation process for producing short-chain alcohols, such as methanol and ethanol, from fermentation of grains and vegetables, such as sugar cane, molasses, corn, potato, barley, wheat or rye.

The C1-C4 alcohol may be ethanol. The conversion of ethanol, e.g. corn ethanol, into high value chemicals provides a commercially valuable alternative to its use as a fuel for combustion.

The fermentation process may be coupled directly or indirectly to the reactor used for the process of the first or second aspect. An advantage associated with this feature is that it provides for cost reduction and improved efficiency in the overall process.

The oxygen-containing organic molecule may be introduced into the reactor in a feed stream having a water content of 30 volume % or more. The water content may, in some embodiments, be 50 volume % or more, such as 80 volume % or more. Waste streams obtained from biological fermentation processes typically have a high water content. Normally, feed streams having such high levels of water are unsuitable for use in processes using an aluminosilicate catalyst. However, an advantage of the process described herein is that it is compatible with feed streams having a high water content without negatively affecting the aluminosilicate catalyst. The process conditions ensure that the water is kept in the vapor phase during the process, eliminating the negative impacts on the catalyst, while simultaneously aiding in removing heavy hydrocarbon liquid deposits from the catalyst surface via vaporization.

The aluminosilicate of the catalyst may have a SiO2:AlO3 ratio of between 20 and 50. Catalysts falling within this range experience minimal degradation when used in the process.

The catalyst may be a zeolite catalyst, preferably ZSM-5 or ZSM-11. Zeolite catalysts, and particularly ZSM-5 and ZSM-11, provide for optimal conversion of CO2 and H2 into the monocyclic aromatic compound. Alternatively, the catalyst may be a combination of a zeolite catalyst and a mixed metal oxide catalyst.

The catalyst may be a metal-modified zeolite catalyst. The metal of the metal-modified zeolite catalyst may be selected from a group VIII, a group 11 or a group 12 metal. Modification of zeolite catalysts by metals in these groups enhances the catalyst activity, resulting in a higher product yield by comparison with non-metal-modified zeolite catalysts.

The oxygen-containing organic molecule is selected from C1-C4 alcohols, C1-C4 ethers, and combinations thereof. For example, the oxygen-containing organic molecule may be methanol, ethanol or combinations thereof, preferably ethanol. Similarly, the oxygen-containing organic molecule may be an ether, such as diethyl ether.

The monocyclic aromatic compound may be selected from the group consisting of benzene, toluene, xylene and combinations thereof.

The present invention will be better understood in light of the following Examples and the accompanying Figures, which are provided for illustrative purposes only and should not be interpreted in a restrictive manner.

### BRIEF DESCRIPTION OF THE FIGURES

In the accompanying Figures:

FIG. 1 is a graph depicting the beneficial effect of a carbon monoxide feed on extending catalyst lifetime.

FIG. 2 is a table showing the total product composition of Example 1 and Example 3 at time on stream of 100 hours.

FIG. 3 is a table showing product selectivity of Example 5. Ethanol conversion in these examples is 100%.

FIG. 4 shows a schematic of an exemplary process for high-selectivity conversion of an oxygen-containing organic molecule to at least one monocyclic aromatic compound.

FIG. 5 shows an embodiment of the process of FIG. 4, including a regeneration process 300.

### DETAILED DESCRIPTION

As used herein and in the accompanying claims, unless the context requires otherwise, the terms below are intended to have the following definitions.

“Comprise” or variations such as “comprises” or “comprising” will be understood to imply the inclusion of a stated integer or group of integers but not the exclusion of any other integer or group of integers.

Unless specifically stated otherwise or obvious from context, as used herein, the term “about” will be understood to fall within a range of normal tolerances in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value, for example ±5%. Unless otherwise clear from the context, all numerical values provided herein are modified by the term “about.”

The monocyclic aromatic compounds described herein includes BTX (benzene, toluene, and xylene isomers, and mixtures thereof). The monocyclic aromatic compounds may include ethylbenzene, and the mixture may then be referred to as BTEX. Xylene isomers may comprise o-xylene, m-xylene, p-xylene, or combinations thereof.

As used herein, “oxygen-containing organic molecule” will be understood to mean an organic molecule having at least one oxygen atom, at least one carbon atom, and at least one hydrogen atom. Preferably, the oxygen-containing organic molecule will be an aliphatic hydrocarbon having from 1 to 4 carbon atoms and one or more oxygen atoms. For example, the oxygen-containing organic molecule may be selected from C1-C4 alcohols, C1-C4 ethers, or C1-C4 esters, and combinations thereof, such as methanol, ethanol, propanol, isopropanol, butanol, isobutanol, or combinations thereof, preferably ethanol, or diethyl ether, methylpropyl ether, or combinations thereof, or methyl formate, ethyl formate, methyl acetate, ethyl acetate, methyl propionate, or combinations thereof.

The term “catalyst” refers to a dehydroaromatization catalyst utilized for the conversion of oxygen-containing organic molecules into monocyclic aromatic compounds. The catalyst is a porous aluminosilicate or zeolitic material with a portion of its pores in the micro-, meso- and/or macro-range. The catalyst may be a zeolite catalyst having a pentasil structure. The zeolite catalyst may be ZSM-5 or ZSM-11. The catalyst may be a combination of a zeolite catalyst and a mixed metal oxide catalyst. Such bifunctional or tandem catalysts, having a combination of a zeolite catalyst and a mixed metal oxide catalyst, have been reported in the literature (for example, in Nezam, Iman, Zhou, Wei, Gusmão, Gabriel S., Realff, Matthew J., Wang, Ye, Medford, Andrew J., and Jones, Christopher W. Direct aromatization of CO2 via combined CO2 hydrogenation and zeolite-based acid catalysis, Journal of CO2 Utilization, Volume 45, 2021, 101405, ISSN 2212-9820), wherein the metal oxides contain the active phase for carbon monoxide and hydrogen activation, and zeolites provide the active sites for C—C coupling. These bifunctional catalysts represent an example of engineering alternate reaction pathways. The zeolite catalyst may be a metal-modified zeolite catalyst. The metal of the metal-modified zeolite catalyst may be selected from a group VIII, a group 11 or a group 12 metal. The SiO2:AlO3 ratio of the zeolite may vary between 20 and 50. Zeolites such as ZSM-5 may be capable of converting oxygen-containing organic molecules into monocyclic aromatic compounds such as BTX via a complex sequence of oligomerization, isomerization, cracking and cyclization reactions that are believed to initiate on Bronsted acid sites of the zeolite. The catalyst may be promoted or unpromoted. Promoting a catalyst is known in the art, and also referred to as loading. This is a well-known procedure which typically involves impregnating or ion-exchanging the catalyst with soluble salts of the promoting elements. The catalyst may be a heterogeneous catalyst comprising aluminosilicate in the range between about 1% to about 99% and preferably 75-99% with an amorphous silica or amorphous alumina, or a combination thereof, in a range of about 0% to about 99% and preferably 1-25%. The aluminosilicate may contain at least 10% and preferably greater than 20% of its total porosity having a mean pore diameter of less than 20 nm. The catalyst may have a total surface area of at least 90 m2/gram and preferably between 90-250 m2/gram. The catalyst may be subject to a regeneration process, wherein the regeneration process comprises the introduction of inert gas and/or an oxidant and/or a reductive fluid at elevated temperature into the reactor.

As used herein, the “weakly-coordinating compound” reduces the H—O bond frequency of the zeolite catalyst or aluminosilicate catalyst framework by about 1 to 300 cm-1 as measured by FT-IR. The weakly-coordinating compound may be a Lewis base. The weakly-coordinating compound may be a labile compound. Non-limiting examples of weakly-coordinating compounds include carbon monoxide, acetonitrile, dimethylsulfoxide, tetrahydrofuran, acetone, pyridine or tetrahydrothiophene.

In arriving at the present invention, it was unexpectedly found that one way to prevent premature catalyst deactivation due to coke formation on the catalyst surface when operating at temperatures that favour high-selectivity of monocyclic aromatic compounds from oxygen-containing organic molecules, i.e., above 400° C., is to contact the catalyst with a weakly-coordinating compound. In theory, a weakly-coordinating compound will coordinate with the most acidic sites of the catalyst and inhibit formation of the coke precursors, thus extending the cycle time of the catalyst. It has also unexpectedly been found that carbon dioxide is able to react with hydrogen gas produced in the catalytic conversion of the oxygen-containing organic molecule to the monocyclic aromatic compounds. The CO2 and H2 react in a reverse water gas shift reaction to produce carbon monoxide and water. The carbon monoxide is a weakly-coordinating compound which can help to prolong the catalyst lifespan. The carbon monoxide can also be converted into additional monocyclic aromatic compounds to increase the yield of the process.

The present disclosure provides a process for manufacturing at least one monocyclic aromatic compound, wherein the process comprises contacting an aluminosilicate catalyst with an oxygen-containing organic molecule in a reactor to produce the monocyclic aromatic compound and in situ-generated hydrogen gas. Three moles of hydrogen gas are generated per mole of monocyclic aromatic compound in the dehydroaromatization reaction of the oxygen containing organic molecule. The process further comprises introducing CO2 into the reactor and allowing the CO2 to react with the in situ-generated hydrogen gas to form additional monocyclic aromatic compound.

At least a portion of the CO2 and in situ-generated hydrogen gas react in a reverse water gas shift process to form carbon monoxide and water. At least a portion of the carbon monoxide may be converted to the additional monocyclic aromatic compound.

The present disclosure also provides a process for manufacturing at least one monocyclic aromatic compound in which the process comprises contacting an aluminosilicate catalyst with an oxygen-containing organic molecule in a reactor to produce the monocyclic aromatic compound and hydrogen gas, introducing CO2 into the reactor, allowing the CO2 to react with the in situ-generated hydrogen gas to form carbon monoxide, and allowing the carbon monoxide to contact the catalyst.

The process may further comprise adding a further weakly-coordinating compound to the reactor. The further weakly-coordinating compound may be selected from the group consisting of carbon monoxide, acetonitrile, dimethylsulfoxide, tetrahydrofuran, acetone, pyridine and tetrahydrothiophene.

The carbon monoxide produced in the reverse water gas shift process, and/or added to the reactor, may be present in the reactor in an amount ranging from 0.1% by weight to 25% by weight, for example, in an amount ranging from 0.1% by weight to 10% by weight, or in an amount ranging from 3% by weight to 7% by weight, based on the overall weight of the components in the reactor. The carbon monoxide may, for example, be present in an amount ranging from 5% by weight to 7% by weight. The carbon monoxide may prolong the cycle time of the catalyst by about 15%.

The CO2 introduced into the reactor may be at least 50%, 60%, 70%, 80%, 90%, 95%, 97%, 98%, 99%, 99.9% or 100% pure. The CO2 may be diluted with atmospheric air before or during introduction into the reactor. Alternatively, undiluted CO2 may be used.

The temperatures in the reactor (e.g., at the input of the reactor, output of the reactor, or intra-reactor) may be within a range of from about 300° C. to about 700° C., from about 350° C. to about 600° C., or from about 400° C. to about 500° C. The temperature may be above 425° C., above 450° C., above 475° C., and/or in a range of from about 450° C. to about 500° C. The pressure within the reactor may be within a range of from about 50 psi to about 500 psi, from about 75 psi to about 250 psi, from about 90 to about 150 psi, from about 100 psi to about 150 psi, or from about 100 psi to about 125 psi. Furthermore, the reaction conditions may include a weight hourly space velocity (WHSV) of from about 0.01 to 10/hour, from about 0.1 to about 5/hour, from about 0.2 to about 2/hour, or from about 0.5 to about 1/hour. The WHSV is defined as the weight of feed flowing per unit weight of the catalyst per hour. Reactor pressure impacts conversion due to the Langmuir Adsorption Isotherm (LAI). In particular, the LAI states that the adsorption of a molecule on a catalyst surface is proportional to the pressure/temperature. As such, as temperature increases, adsorption decreases and to overcome this, pressure must increase. Thus, to increase conversion yield of monocyclic aromatic compound per converted oxygen-containing molecule, the pressure within the reactor must be increased. However, this increase results in lower cycle times of the catalyst in the reactor. Accordingly, the present application provides an improvement by adding a weakly coordinating base to the reaction so that the cycle time of the catalyst increases.

The CO2 used in the processes of the invention may be derived from a biological, combustion, or chemical transformation process. For example, the CO2 may be obtained from biological sources (such as fermentation processes), combustion processes (such as coal and gas fired power stations), or industrial processes (such as fertilizer or cement production facilities).

The oxygen-containing organic molecule may be derived from a fermentation process. The fermentation process may be a process for producing a C1-C4 alcohol (e.g. methanol, ethanol, etc) by fermentation of grains and vegetables, such as sugar cane, molasses, corn, potato, barley, wheat and rye. Preferably, the CO2 and oxygen-containing organic molecule are derived from the same fermentation process and coupled, either directly or indirectly, with the reactor to provide the input streams for the processes of the invention. The CO2 may be captured, compressed, and then introduced into the reactor. For example, CO2 obtained from alcohol production, which is typically pure (>99.9%), can be relatively easily compressed and injected into the reactor.

The output streams of fermentation processes, such as the output streams of bioethanol production, have a high water content. While feedstocks having a high water content are normally detrimental to catalytic processes involving zeolite catalysts, the processes of the present disclosure are capable of converting feed streams of alcohols with high water contents into the monocyclic aromatic products without substantial detriment to the catalyst. Therefore, the oxygen-containing organic molecule may be introduced into the reactor in a feed stream having a water content of 30 volume % or more, 50 volume % or more, or 80 volume % or more. It has been surprisingly found by the present inventors that increased water contents in the feedstocks are capable of leading to higher yields of monocyclic aromatic products in the processes of the invention. This is contrary to expectations based on the prior art, since higher water contents would be expected to de-aluminate zeolite catalysts, with consequential deleterious effects on both catalyst life and product yield.

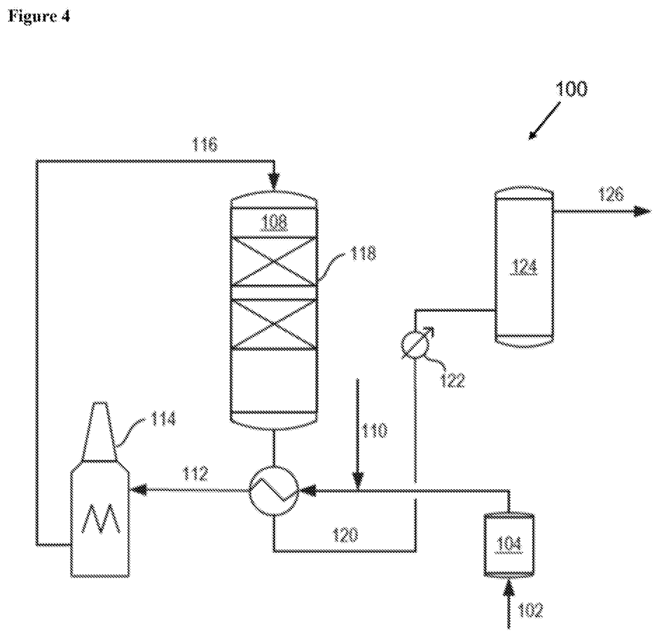

Referring to the Figures, FIG. 4 depicts an exemplary schematic 100 for the high-selectivity conversion of an oxygen-containing organic molecule to a monocyclic aromatic compound. A gas feed containing the oxygen-containing organic molecule enters the process as stream 102 and may include a stream from a fermentation process, which may comprise C1-C4 alcohols. Stream 102 may be obtained directly or indirectly from a fermentation process.

Stream 102 may be sent through exchangers and heaters 104 prior to reaction. Prior to entry to a reactor 108, an additive stream 110 introduces a weakly-coordinating compound or precursor thereof to stream 102 prior to reaction resulting in a combined stream 112 that is input into reactor 108. In an embodiment, the reactor 108 is a fixed bed, fluidized bed, or moving bed. In an embodiment, the reactor 108 is a catalytic reactor, a gasifier, or a pyrolysis reactor.

In embodiments, the combined stream 112 may be heated by a fired heater 114 prior to entry into reactor 108 resulting in a hot feed 116. The hot gas feed 116 enters reactor 108 and is reacted over the catalyst 118 bed(s) containing a dehydroaromatization catalyst. In an embodiment, the stream of weakly-coordinating compound or precursor thereof introduced via additive stream 110 reduces the H—O bond frequency of the catalyst 118 (e.g., a zeolite or aluminosilicate framework) by about 1 to 300 cm-1 as measured by FT-IR.

After the reaction, the resulting product stream is output from the reactor 108 as output stream 120 and is cooled using cooler 122. Cooler 122 is optional. The output stream 120, containing two-phase liquid and vapor products are separated in vessel 124. The resulting products exit as a separated product stream 126.

Although shown as being added to the stream 102, the additive stream 110 may be introduced at any point prior to entry of the hot gas feed 116 into the reactor 108. In one embodiment, the additive stream 110 may be introduced to stream 102 after stream 102 is heated by fired heater 114. Furthermore, the additive stream 110 may be introduced directly to the reactor 108 as a separate stream to the hot feed 116.

In an embodiment, reactor 108, during conversion of the oxygen-containing organic molecule to the monocyclic aromatic compound, is subjected to a temperature of >450° C., >500° C., and/or >550° C., in an outlet or inlet of reactor 108, or intra-reactor.

In an embodiment, reactor 108, during conversion of a first composition comprising the oxygen-containing organic molecule to the monocyclic aromatic compound, is subjected to a pressure of 50 psi to about 500 psi.

The high-selectivity conversion of the oxygen-containing organic molecule to the monocyclic aromatic compound achieved via the process shown in schematic 100 is >25%, >45%, >65%, >75%, >85%, >95%, preferably >65%.

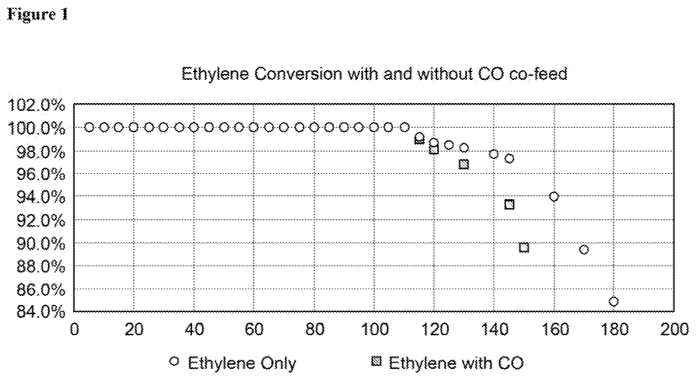

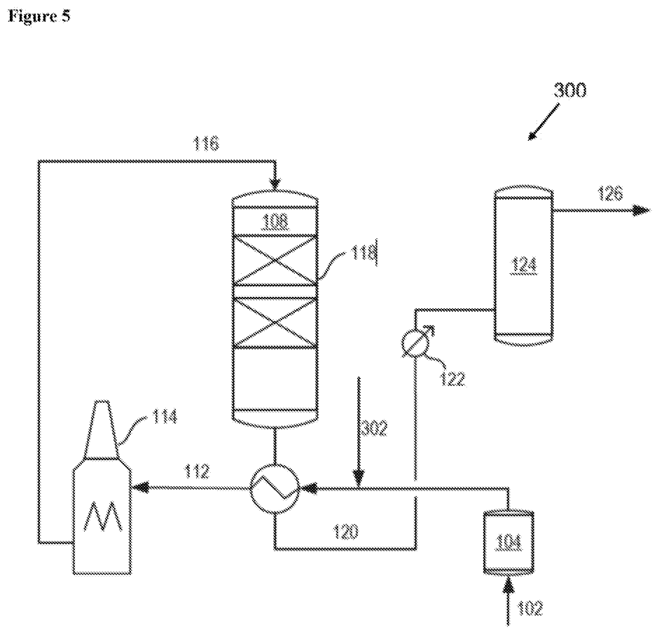

FIG. 5 shows an embodiment of the process of FIG. 1, including a catalyst regeneration process 300. In an embodiment, the catalyst 118 is subject to a regeneration process, wherein said regeneration process comprises the introduction of regeneration compound 302 and operating the reactor 108 at a regeneration temperature. In one example, the regeneration compound 302 is one or more of an inert gas, an oxidant, a reductive fluid, and any combination thereof. In an embodiment, operating the reactor 108 at a regeneration temperature includes gradually increasing the temperature in the reactor 108 to >450° C. over a regeneration period. In some embodiments, the temperature of reactor 108 during the regeneration process is increased to between 300° C. and 700° C., preferably from 400 to 550° C., or from 450 to 500° C. during the course of the regeneration process. In some embodiments, the temperature is increased to about 500° C. In some embodiments, the oxidant is provided at low concentrations (e.g., about 1% oxygen gas in nitrogen gas) and increased over the course of the regeneration process.

Thus, in instances of catalysts with a high degree of coke formed in/on the catalyst, rapid introduction of an oxidant may cause an excessive exotherm which can damage the catalyst. In these instances, it may be favourable to slowly introduce either an inert gas or oxidant at lower temperatures and increase temperature accordingly to maintain temperatures favourable for catalyst regeneration but not too severe to negatively impact catalyst structure. Accordingly, the above temperature ranges and concentration of the introduced regeneration compound 302 may be based on the amount of coke formation, catalyst type, and other factors that relate to catalyst damage during the regeneration thereof.

Rapid deactivation of zeolite catalysts (either by coke formation at high temperature operation and/or high water content in the feed stream) is economically unfavourable if the catalyst is not, or cannot be regenerated. In the high temperature conversion of monocyclic aromatic compounds from oxygen-containing organic molecules, as much as 20 to 25 wt % carbon can accumulate on the surface and inside the pores of the catalyst upon deactivation. Regeneration of the catalyst and removal of both the coke formed in/on the zeolite catalyst can be accomplished through the use of an oxidant, e.g. air, N2/O2 or H2O2 allowing for extended catalyst lifetime.

### EXAMPLES

### Example 1

10 grams of 16 mesh Zn modified ZSM5 catalyst charged in a 0.500 inch od×0.035 inch wall 316SS reactor was heated to 500° C. under 5.0 liters per hour nitrogen at 100 psig. Once the temperature stabilized, 1.5 liters per hour of ethylene was introduced into the nitrogen feed. The entire reactor product was analyzed on-line using an Agilent 7890B GC equipped with a 100 meter DHA boiling point column and FID to monitor ethylene conversion and product selectivity. The reaction was stopped after 150 hours when ethylene conversion was observed to drop below 90%.

### Example 2

To show that a weakly coordinating species has a beneficial effect in extending catalyst life, Example 1 was repeated by replacing nitrogen with 10 mole % carbon monoxide in nitrogen. The reaction was stopped after 170 hours, a 13% life extension, when ethylene conversion was observed to drop below 90%. FIG. 1 shows ethylene conversion versus time on stream comparison of Example 1 and Example 2 and shows that addition of carbon monoxide resulted in a 20 hour ethylene conversion extension, thereby highlighting the beneficial effect that a weakly coordinating compound has on extending the catalyst life.

### Example 3

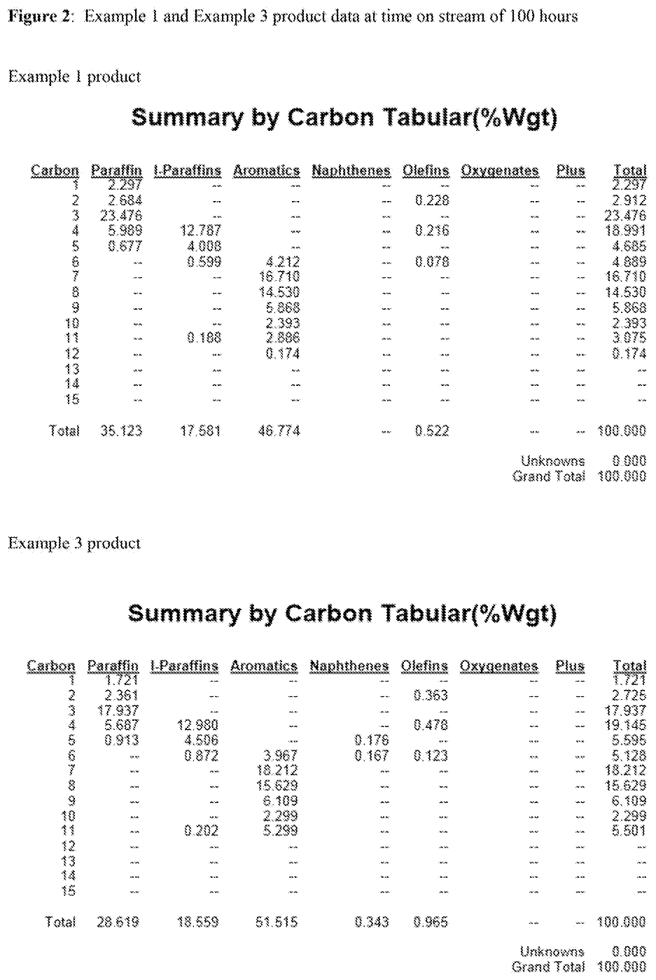

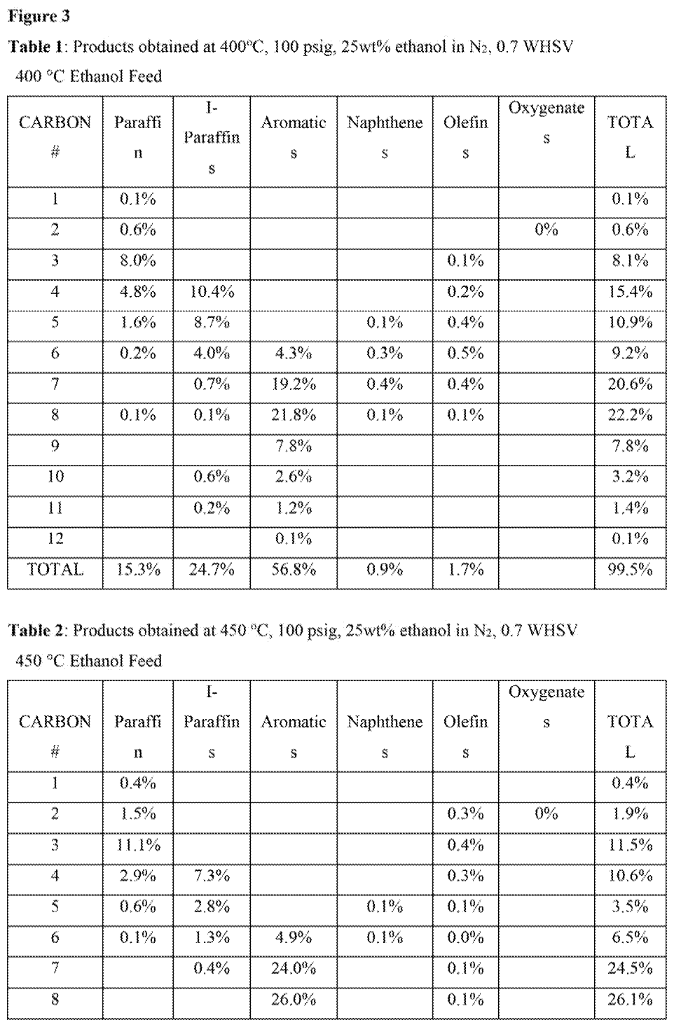

To show the beneficial effect of co-feeding carbon dioxide which subsequently reacts with the in-situ hydrogen produced in the dehydroaromatization reaction, Example 1 was repeated by replacing nitrogen with 10 mole % carbon dioxide in nitrogen. The entire reaction product was analyzed on-line using an Agilent 7890B GC equipped with a 100 meter DHA boiling point column and FID to monitor ethylene conversion and product selectivity. FIG. 2 shows the total product composition of Example 1 versus Example 3 at time on stream of 100 hours.

### Example 4

10 grams of a zeolite catalyst charged in a 0.500 inch od×0.035 inch wall 316SS reactor was heated to 400-600° C. under 5.0 liters per hour nitrogen at 50-150 psig. Once the temperature stabilized, 5-20 mole % CO2/H2 was introduced. The entire reactor byproduct was analyzed on-line using an Agilent 7890B GC equipped with a 100 meter DHA boiling point column and FID and TCD to monitor the formation of hydrocarbons and non-flammable light gases, including aromatic hydrocarbons.

### Example 5

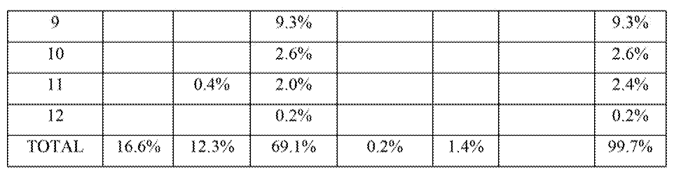

10 grams of 16 mesh Zn modified ZSM5 catalyst charged in a 0.500 inch od×0.035 inch wall 316SS reactor was heated through 400 C to 450 C under 5.0 liters per hour nitrogen at 100 psig. Once the temperature stabilized, 1.93 g/hr anhydrous ethanol per hour was introduced into the nitrogen feed. The entire reactor product was analyzed on-line using an Agilent 7890B GC equipped with a 100 meter DHA boiling point column and FID to monitor ethanol conversion and product selectivity. FIG. 3 shows the ethanol conversion and product selectivity.

1. A process for manufacturing at least one monocyclic aromatic compound, the process comprising contacting an aluminosilicate catalyst with an oxygen-containing organic molecule in a reactor to produce the monocyclic aromatic compound and in situ-generated hydrogen gas; and introducing CO₂ into the reactor and allowing the CO₂ to react with the in situ-generated hydrogen gas to form additional monocyclic aromatic compound.

2. The process of claim 1, wherein at least a portion of the CO₂ and in situ-generated hydrogen gas react to form carbon monoxide, and wherein the process further comprises allowing the carbon monoxide to contact the catalyst.

3. A process for manufacturing at least one monocyclic aromatic compound, the process comprising contacting an aluminosilicate catalyst with an oxygen-containing organic molecule in a reactor to produce the monocyclic aromatic compound and in situ-generated hydrogen gas, introducing CO₂ into the reactor, allowing the CO₂ to react with the in situ-generated hydrogen gas to form carbon monoxide, and allowing the carbon monoxide to contact the catalyst.

4. The process of claim 3, wherein at least a portion of the CO₂ and in situ-generated hydrogen gas react to form additional monocyclic aromatic compound.

5. The process of claim 1, further comprising adding a further weakly-coordinating compound to the reactor, wherein the weakly-coordinating compound is a compound that reduces the H—O bond frequency of the aluminosilicate catalyst by about 1 to 300 cm-1 as measured by FT-IR.

6. The process of claim 5, wherein the weakly-coordinating compound is selected from the group consisting of carbon monoxide, acetonitrile, dimethylsulfoxide, tetrahydrofuran, acetone, pyridine and tetrahydrothiophene.

7. The process of claim 1, wherein the CO₂ is derived from a biological, combustion, or chemical transformation process.

8. The process of claim 1, wherein the oxygen-containing organic molecule is derived from a fermentation process.

9. The process of claim 8, wherein the fermentation process is a process for producing a C₁-C₄ alcohol from a grain crop, such as corn.

10. The process of claim 9, wherein the C₁-C₄ alcohol is ethanol.

11. The process of claim 8, wherein the fermentation process is coupled directly or indirectly to the reactor.

12. The process of claim 1, comprising introducing the oxygen-containing organic molecule into the reactor in a feed stream having a water content of 30 volume % or more.

13. The process of claim 12, wherein the feed stream has a water content of 50 volume % or more, such as 80 volume % or more.

14. The process of claim 1, wherein the aluminosilicate has a SiO₂:AlO₃ ratio of between 20 and 50.

15. The process of claim 1, wherein the catalyst is a zeolite catalyst, preferably ZSM-5 or ZSM-11 or a combination of a zeolite catalyst and a mixed metal oxide catalyst.

16. The process of claim 1, wherein the catalyst is a metal modified zeolite catalyst.

17. The process of claim 16, wherein the metal of the metal modified zeolite catalyst is selected from a group VIII, 11 or 12 metal.

18. The process of claim 1, wherein the oxygen-containing organic molecule is selected from C₁-C₄ alcohols, C₁-C₄ ethers, and combinations thereof.

19. The process of claim 18, wherein the oxygen-containing organic molecule is ethanol.

20. The process of claim 1, wherein the monocyclic aromatic compound is selected from the group consisting of benzene, toluene, xylene and combinations thereof.
